(19) United States
(12) Patent Application Publication
(10) Pub. No.: US 2010/0304418 A1
(43) Pub. Date: Dec. 2, 2010

(54) Compositions and Methods for Monitoring Flow Through Fluid Conducting and Containment Systems

(76) Inventors: Artin Moussavi, London (GB); Catherine Rowley-Williams, Linuthgow (GB); Cameron MacKenzie, Glasgow (GB); Fiona MacKay, Edinburgh (GB); Anne-Marie Fuller, Edinburgh (GB); Vjera Magdalenic, London (GB); Emma Perfect, Edinburgh (GB)

Correspondence Address: OSTRAGER CHONG FLAHERTY & BROITMAN PC, 570 LEXINGTON AVENUE, FLOOR 17, NEW YORK, NY, 10022-6894, US

(21) Appl. No.: 12/808,387

(22) PCT Filed: Dec. 17, 2008
(86) PCT No.: PCT/GB2008/004174

§ 371 Date: Aug. 9, 2010

(30) Foreign Application Priority Data
Dec. 17, 2007 (GB) 0724540.0
Oct. 7, 2008 (GB) 0818359.2

(51) Int. Cl. C12Q 1/28; C07D 495/02; C07H 3/02; C07D 473/06; C07D 473/30; G01N 31/00

(52) U.S. Cl. 435/28; 548/304.1; 536/1.11; 544/267; 544/265; 436/2

(57) ABSTRACT

Latently detectable tracers for use in fluid conducting and containment systems wherein the interaction between the tracer and a biomacromolecule produces a detectable signal. More specifically, the latently detectable tracers monitor flow through such systems. Methods for monitoring the flow of fluid using the tracers, and a kit for use in monitoring the flow of fluid in such systems, including the tracers are also disclosed.

This invention relates to latently detectable tracers for use in fluid conducting and containment systems. More specifically, the invention relates to latently detectable tracers for monitoring flow through such systems, methods for monitoring the flow of fluid using the tracers, and a kit for use in monitoring the flow of fluid in such systems, including the tracers.

Fluid conducting and containment systems are susceptible to inefficiencies and loss of productivity due to damage of component parts. For example, oil and gas operators continue to lose millions of barrels of potential oil production each day due to corrosion, scale and hydrate build up and microbial growth. Such systems include, for example, oil and gas reservoirs, petrochemical processing facilities, refineries, paper manufacture, mining, cooling towers and boilers, water treatment facilities and also natural and man-made water systems e.g. lakes, reservoirs, rivers, and geothermal fields.

Keeping equipment, pipes and other infrastructure healthy is ultimately the most efficient way to ensure maximum production and efficiency. The fluid conducting and containment portions of such systems must be continually monitored as many factors can reduce flow efficiency, for example, corrosion of pipes and build up of microbial growth, scale, hydrates, asphaltenes and waxes. Monitoring of natural water systems is also important, for example to provide information on the flow of water from different sources, to assess the environmental impact of certain processes and to gather information relating to currents. Detectable moeties can be used to monitor the efficiency of flow of fluid and specific components of fluid in systems. Applications include, but are not limited to, investigation of leaks, speed of flow and how fluid from different systems becomes mixed.

The frequency of chemical interventions is a critical cost factor. Despite the chemical interventions in place many losses are still incurred, for example, oil and gas operators continue to lose millions of barrels of potential oil production each day due to corrosion, scale and hydrate build up and microbial growth. Information gathered during monitoring of fluid flow can be used to ensure the effective deployment of interventions, which help maintain asset integrity and optimal flow in a system. For example the use of so-called “treatment substances”, to help maintain flow efficiency. Such treatment substances may include scale inhibitors, corrosion inhibitors, hydrate inhibitors, wax inhibitors, anti-fouling agents, asphaltene inhibitors, pH stabilisers, hydrogen sulfide scavengers, flow additives, anti-foaming agents, detergents and demulsifiers. Such treatment substances may be used in oil and gas wells, oil and gas pipelines, petrochemical processing plants, paper manufacture, mining, cooling towers, boilers, water treatment facilities and natural water courses. The term a “treatment substance” is not intended to be limited in the substances to which this patent application refers.

There is therefore a clear need to monitor the flow of fluid in both industrial fluid conducting and containment systems and natural water systems. This monitoring process can be labour-intensive and expensive, especially but not limited to cases requiring the offshore monitoring of flow of fluid used in sites such as oil wells. For the latter, samples are often flown onshore for testing, which is especially expensive and time consuming. As fields mature, flights to shore become less frequent, resulting in less comprehensive testing. Risks of well failure are therefore increased and the need for simple offshore testing grows. In general, there is a need for cost-effective, simple, convenient on-site sample testing methods and compositions for use in such methods, in order to measure flow of fluid throughout fluid conducting and containment systems.

In particular, being able to monitor fluid flow would allow the early detection of flow assurance problems and the implementation of preventative action to minimise the risks of production loss. For example, the kinds of objectives that could be achieved by monitoring fluid flow could include quantifying the volume of water, oil, or other fluid flowing in a system; quantifying the speed of fluid flowing in a system; determining the preferential flow trends of a system such as a reservoir; determining the injector used to produce flow relationships; investigation of leaks in a system, and the determination of how fluid from different systems become mixed for example how water, injection or produced, from different wells becomes mixed. Preventative action taken after obtaining this information may include, for example, early planning of squeeze treatments; informing the application of treatment substances in response to flow assurance problems in pipelines, and maximising efficiency of usage of treatment substances so they are only added when required ie when specific flow problems have been detected.

A useful method to monitor the flow of fluid is to use a detectable moiety whose movement can be predicted and monitored and used to obtain information about a system. These detectable moieties may also be called “tracers”. Many systems are suitable for monitoring with tracers. These may be industrial, for example downhole or formation region of drilling site, or well bore region of a formation, or natural, such as watercourses. Tracers are currently used to monitor the flow of fluid and specific components of fluid in systems. Such tracers include chemicals, such as salts of various types including potassium chloride; inert gases, such as krypton or xenon; various hydrocarbon compounds; coloured chemicals and fluorescent chemicals such as fluorescein and rhodamine. Radioactive materials may also used, such as deuterium oxide and tritium. As an example of the use of radioactive tracers see U.S. Pat. No. 5,077,471, in which radioactive tracers are used to indicate the fluid flow from the formation. Both deuterium oxide and tritium are effective radioactive tracers, but both are relatively expensive and subject to strict import and export restrictions because of their radioactivity. Chemical tracers have also been used. These tend to be less restricted for usage since they are not radioactive, although can be expensive, may have solubility issues, may not be detectable at sufficiently low concentrations and may be degraded, particularly under the harsh conditions in oil and gas wells.

WO 2005/000747, U.S. Pat. No. 6,312,644, U.S. Pat. No. 5,621,995 and U.S. Pat. No. 5,171,450 describe the use of fluorescently detectable moieties as conjugated tracers for scale inhibitors and other water treatment chemicals. However, fluids used in such systems may be dark coloured e.g. dark oil and so may mask the signal from fluorescent or coloured tracers. Alternatively the fluids may be highly fluorescent e.g. corrosion inhibitors, oil or algae, and therefore the signal-to-background ratio can be poor, necessitating complicated data processing to detect the tracer. It would be preferable to have a moiety for use in monitoring fluid flow that is only latently detectable by a chosen method of detection on addition of a reagent. Furthermore, these patents do not disclose the use of fluorescent tracers free in a fluid; rather the moieties are attached to a water treatment chemical.

U.S. Pat. No. 6,040,406 describes a polymerisable, latently detectable moiety which is converted by a photoactivator into a moiety that absorbs light within a wavelength from 300 to 800 nm. In other words, the method of detection for this moiety is colourimetry, in which a colour change in a sample indicates the presence and concentration of the moiety. Colourimetry is not always appropriate as a method of detection, for example if it is required that a signal from a coloured or opaque sample such as oil or contaminated water be measured. In order to ensure that many different types of sample can be tested, it would therefore be preferable to have a range of latently detectable moieties, each of which is detectable using a number of different methods that do not suffer from the problems of low visibility due to background signals.

U.S. Pat. No. 6,218,491 and U.S. Pat. No. 6,251,680 describe water-soluble polymers having amine-thiol terminal moieties incorporated for the attachment of an amine-reactive detectable label. The detectable label is added to a sample taken from a body of fluid in order to analyse the concentration of the water-soluble polymer. The amine-thiol terminal moieties are various derivatives of peptides and polypeptides. The problem with the use of such molecules as labels for treatment substances or as tracers to monitor flow is that under the extreme conditions encountered within oil and water treatment facilities, amino acid polymer-based molecules are unstable. There remains a need for latently detectable tracer that are robust to the harsh environment of such industrial systems.

Tracers comprising salts have also been used. For example, WO2007102023 describes the use of non-radioactive metals and their salts. Such tracers can have low detection limits although the technology required to detect the tracers such as in produced fluids requires highly skilled personnel and expensive equipment such as inductively-coupled plasma-mass spectroscopy (ICP-MS).

In summary, what is needed in the art are detectable tracers which are chemically and thermally stable, cost effective, acceptably safe i.e. non toxic, not flammable, not corrosive, not radioactive, not susceptible to sample interferences, simple to detect with high specificity, and can be detected at very low concentrations, preferably <1 part per million. There remains a need for a tracer that can be used for monitoring of fluid flow in industrial and/or natural conducting and containing systems. It would be preferable if such tracers and methods of using the tracers were sufficiently adaptable and simple so that monitoring could be performed online, atline, inline or offline. Preferably, the tracers and any method of using them would have minimal deleterious impact on the system being investigated. Such systems may include an oil well, gas well, hydrocarbon flow line, refinery, factory or river system. For oil and gas applications it is desirable that the tracers and reaction methods are robust to the harsh environment of the oil well, including high temperatures, high pressures, presence of treatment chemicals, oil and high ionic strength solutions. Finally, it would be beneficial to provide tracers that are easily detectable in a sample, so that any problems of poor signal to background ratio may be addressed.

It is an object of the present invention to provide compositions that seek to address the problems highlighted above.

### DEFINITIONS

A “tracer” is defined for the purposes of this description as a moiety that interacts specifically with an associated biomacromolecule. The tracer may be latently detectable, producing a detectable signal only on interaction with said associated biomacromolecule.

“Latently detectable” is used within this description to mean that a tracer is not detectable by a chosen method of detection, until it interacts with the recognition site of a biomacromolecule. The interaction results in a change in the sample, or a change in the biomacromolecule, which can be detected by the chosen method of detection.

A ‘fluid conducting and containment system’ or ‘system for conduction and containment of fluid’ or ‘fluid system’ refers to any such system that is used in or by industry. This may include natural water systems. The term may also mean those systems used in industries for which efficiency of flow is important in order to achieve high productivity or to maximise effectiveness. The term may also refer to any system that is treated by treatment substances, the treatment substances being used to enhance flow efficiency within the system. Such treatment substances are discussed within this patent application. Examples of such fluid conducting and containment systems that would benefit from the present invention include oil and gas reservoirs and their associated infrastructure (wells, pipelines, separation facilities etc), petrochemical processing facilities, refineries, paper manufacture, mining, cooling towers and boilers, water treatment facilities and natural water systems e.g. lakes, reservoirs, rivers, and geothermal fields. As would be understood by the skilled person, such systems tend to be large, but may include small components and in addition, some such systems may be small, such as microfluidic devices.

A “biomacromolecule” is defined for the purposes of this description as a biomacromolecule e.g. protein, that includes a site for the specific interaction, binding or displacement of a small molecule, of which a number of non-limiting examples are listed in Table 1. This interaction may be based on conformational or chemical aspects of the tracer and/or the biomacromolecule. This may also include the binding or interaction of a tracer with a ligand that is already associated with the biomacromolecule, for example displacement of the ligand by the label. The biomacromolecule may be adapted to produce a signal on binding of the tracer, or it may do so due to an innate, pre-existing property of the biomacromolecule. This signal may be chemical, for example production of hydrogen peroxide, or the signal may be light-based. For example a fluorophore could be attached to a biomacromolecule, such as a molecule of streptavidin. Alternatively, the biomacromolecule may produce a signal due to a pre-existing property, for example it may be a photoprotein and emit light, or it may be an enzyme and produce a molecule on interaction with the tracer. Any biomacromolecule known in the art to associate specifically via such a recognition or binding site with a small molecule would fit this definition. The term may include many small molecule-biomacromolecule pairs exist in nature as listed non-exhaustively below:

TABLE 1

| | Biomacromolecule to which the |
|---|---|
| Tracer | tracer binds |
| Biotin | Streptavidin or avidin or neutravidin |
| | or captavidin, also mutant variants |
| | and derivatives of these |
| Selenobiotin | Streptavidin or avidin or neutravidin |
| | or captavidin, also mutant variants |
| | and derivatives of these |
| Oxybiotin | Streptavidin or avidin or neutravidin |
| | or captavidin, also mutant variants |
| | and derivatives of these |
| Thiamine | Thiamine binding protein |
| Riboflavin and Riboflavin-5′- | Riboflavin binding protein |
| phosphate | (flavoprotein) |
| Niacin (nicotinic acid) | Nicotinic acid binding protein |
| Pantothenic acid | Pantothenic acid binding protein |
| Citrate | Citrate binding protein |
| Cobalamin | Cobalamin binding protein |
| Folic acid | Folic acid binding protein |
| Ascorbic acid | Ascorbic acid binding protein |
| Retinol | Retinol binding protein |
| Vitamin D, cholecalciferol and | Vitamin D binding protein e.g. |
| calcitriol | group specific protein (Gc), 25- |
| | hydroxylase, vitamin D receptor, |
| | antibodies (such as from DiaSorin) |
| Vitamin E | Vitamin E binding protein |
| Vitamin K | Vitamin K binding protein |
| Glucose and derivatives including | Glucose binding protein including |
| 2-N-acetyl glucosamine, 1-Methyl- | glucose oxidase |
| beta-D-glucopyranoside, 1-Hexyl- | |
| beta-D-glucopyranoside and | |
| derivatives at position 4. | |
| Fructose | Fructose binding protein |
| Maltose | Maltose binding protein |
| Ribose | Ribose binding protein |
| Other sugars, polysaccharides and | Lectins (various) |
| carbohydrates e.g. arabinose, | |
| deoxyribose, lyxose, ribulose, | |
| xylose, xylulose and starch | |
| Chitin | Chitin binding protein |
| D-Luciferin | Luciferase e.g. firefly luciferase, |
| | railroad worm luciferase, click |
| | beetle luciferase |
| Coelenterazine | Coelenterate luciferases e.g. Renilla, |
| | Gaussia and photoproteins e.g. |
| | aequorin and obelin |
| Histidine | Histidine transporter protein |
| Caffeine | Caffeine binding protein |
| Imidazoline | Imidazoline binding protein |
| Steroid hormones eg cortisol | Steroid hormone receptors eg |
| | cortisol binding protein |
| Chlorpromazine | Chlorpromazine binding protein eg |
| | receptors of central nervous system |
| cAMP | cAMP binding protein |
| cortisol | Cortisol binding protein (reference: |
| | Biology of Reproduction, Vol 18, |
| | 834-842) or cortisol antibody as |
| | used conjugated to luciferase marker |
| | (Sensomics) |
| 6-keto-prostaglandins | 6-keto-prostaglandin antibody, |
| | including labelled antibodies such as |
| | aequorin or GFP labelled versions |
| | available from Senseomics |
| Thyroxine | Thyroxine binding proteins |
| | including thyroxine-binding |
| | globulin, transthyretin and albumin |
| Triiodothyronine | Thyroxine binding proteins |
| | including thyroxine-binding |
| | globulin, transthyretin and albumin, |
| | nuclear Triiodothyronine binding |
| | protein (Proc Natl Acad Sci USA. |
| | 1974 October; 71(10): 4042-4046) |
| Anthocyanins | Glutathione S-transferases |
| Cholesterol | Cholesterol binding proteins such as |
| | VIP21/caveolin and cholesterol |
| | oxidase |
| L-gulono-1,4-lactone | L-gulono-1,4-lactone binding |
| | proteins including: Rv1771, |
| | L-gulono-1,4-lactone dehydrogenase/ |
| | oxidase |
| Bile acids and salts including | glutathione S-transferases, bile acid |
| cholic acid, chenodeoxycholic | binding proteins such as ileal bile |
| acid, deoxycholic and | acid binding proteins, liver fatty |
| glycocholate | acid-binding proteins |
| eicosanoids (prostaglandins, | Prostaglandin receptors e.g. PPARg, |
| prostacyclins, the thromboxanes | Prostacyclin receptors e.g. PTGIR; |
| and the leukotrienes) | thromboxane receptors e.g. TXA2 |
| Vitamin C (L-ascorbate) | L-ascorbate binding protein |
| | including L-ascorbate oxidase |
| Galactose and derivatives including | Galactose binding protein including |
| 2-N-acetyl galactose, 1-Methyl- | galactose oxidase |
| beta-D-galactose and 1-octyl- | |
| beta-D-galactose | |
| Xanthine and hypoxanthine | Xanthine oxidase, xanthine |
| | dehydrogenase, |
| | phosphoribosyltransferase, Xanthine |
| | binding RNAs |
| Catecholamines such as | catecholamine regulated protein |
| epinephrine and norepinephrine | (CRP40), catecholamine binding |
| | proteins, adrenergic receptors (alpha |
| | and beta), epinephrine receptor, |
| | norepinephrine receptor |
| Nucleotides (adenine, cytosine, | Nucleotide binding proteins e.g. G |
| guanine, tyrosine, uracil; | proteins, ATP-binding protein |
| monophosphate, diphosphate and | |
| triphosphate forms) | |

According to one aspect of the present invention, there is provided a tracer for monitoring flow through a system for conduction and containment of fluid, wherein the interaction between the tracer and a biomacromolecule produces a detectable signal. This tracer is ideal for use within fluid conduction and containment systems because it can be easily and conveniently monitored even on-site at off-shore or remote locations by adding a biomacromolecule, and detecting the resulting signal. The user can be sure that any signal that is produced on addition of the biomacromolecule is due to the presence of the tracer, because the biomacromolecule has a high specificity for the tracer. Thus, no signal will be emitted unless the tracer is present. A further advantage is that the tracer is latently detectable. Therefore, the expected signal will not be produced from the fluid, even if it contains the tracer, until the biomacromolecule is added. In order to detect the signal attributable to the presence of the tracer, a signal measurement can be taken before and after addition of the biomacromolecule, and the former subtracted from the latter. This simple subtraction ensures that any interfering background signal can be easily removed. Sometimes it is necessary to treat the sample to remove background interference such as autofluorescence by addition of chemicals, heat treatment or bleaching. If tracers are directly detectable, they may be affected by such treatment and become less detectable—but a latently detectable tracer will advantageously not be affected by such treatment.

Preferably, the biomacromolecule includes a site for specific interaction with the tracer. The biomacromolecule and the tracer may associate as part of molecular signalling complexes in nature. As such, the biomacromolecule is only capable of interacting with the label, so that a signal is only produced if the tracer, and therefore the composition, is present. This allows for extremely precise detection of the presence of the composition, reducing the likelihood of false positive results. Preferably, the biomacromolecule does not have to be added to the fluid conducting and containment system, so that it is not damaged by the harsh conditions typically present in industrial systems.

The detectable signal produced due to the interaction between the tracer and the biomacromolecule may be an optical signal. This may be generated, for example, because the biomacromolecule is conjugated to a fluorophore and the tracer displaces a quencher, so that a fluorescent signal is emitted. Alternatively, the optical signal may be generated directly due to a chemical, conformational or other change in the biomacromolecule, for example if it is a photoprotein that emits light on contact with the label.

The signal may be generated on addition of a second molecule to a sample or fluid containing the tracer and the biomacromolecule. This would be useful, for example, where a chemical change has been produced as a result of the interaction between the biomacromolecule and the tracer.

Preferably, the tracer is a small molecule that is known to interact with a specific biomacromolecule in nature, for example as part of a molecular signalling complex. This may be because the tracer fits into an ‘interaction’ or ‘active’ site within the biomacromolecule and is capable of creating a temporary or permanent interaction with the site. The interaction may be due to ionic or covelant bonds, electrostatic interactions or any other bonds or forces, but should be sufficiently stable that a there is enough time for the signal produced as a result of the interaction to be detected. As such, the tracer is only detected on interacting with the biomacromolecule, so that a signal is only produced if the biomacromolecule is present. This allows for extremely precise detection of the presence of the composition, reducing the likelihood of false positive results.

Preferably, the tracer is selected from: vitamins including biotin, selenobiotin or oxybiotin, thiamine, riboflavin, niacin (nicotinic acid), pathothenic acid, citrate, cobalamin, folic acid, ascorbic acid, retinol, vitamins C, D, E or K; luciferin; coelenterazine; chitin; amino acids such as histidine; or monosaccharides, polysaccharides and carbohydrates including arabinose, deoxyribose, lyxose, ribulose, xylose, xylulose, maltose, glucose, fructose, ribose, or trehalose, caffeine, imidazoline, steroid hormones, chlorpromazine and cAMP, cortisol, 6-ketoprostaglandins, thyroxine, triiodothyronine, anthocyanins, cholesterol, L-gulono-1,4-lactone, bile salts including cholic acid, chenodeoxycholic acid, deoxycholic and glycocholate eicosanoids (prostaglandins, prostacyclins, the thromboxanes and the leukotrienes), galactose and derivatives including 2-N-acetyle galactose, 1-methyl-beta-D-galactose, 1-octyl-beta-D-galactose, xanthine and hypoxanthine, catchetolamines such as epinephrine and norepinephrine, nucleotides such as adenine, cytosine, guanine, tyrosine, uracil, monophosphate, in diphosphate and triphosphate forms and the associated biomacromolecule is selected accordingly to the tracer used from; avidin and its functional analogues e.g. streptavidin, neutravidin and nitroavidin; thiamine binding-protein; riboflavin binding protein (flavoprotein); nicotinic acid binding protein; pantothenic acid binding protein; citrate binding protein, cobalamin binding protein; folic acid binding protein; ascorbic acid binding protein; retinol binding protein; vitamin D binding protein e.g. group specific protein (Gc); Vitamin E binding protein; Vitamin K binding protein; luciferase; coelenterate luciferase; chitin binding protein; histidine transporter protein; arabinose binding protein; deoxyribose binding protein; lyxose binding protein; ribulose binding protein; xylose binding protein; xylulose binding protein; maltose binding protein; glucose binding protein; fructose binding protein; ribose binding protein; trehalose binding protein or lectin; caffeine binding protein; imidazoline binding protein; steroid hormone receptors; chlorpromazine binding protein; cAMP binding protein; cortisol binding protein; 6-keto-prostaglandin antibody including labelled antibodies such as aqueorin or GFP labelled antibodies; thyroxine binding proteins including thyroxine binding globulin, transthyretin and albumin; triiodothronine binding protein; glutathione-S-transferases; cholesterol binding proteins such as VIP21/caveolin and cholesterol oxidase; L-gulono-1,4-lactone binding proteins including Rv1771, L-gulono-1,4-lactone dehydrogenase and L-gulono-1,4-lactone oxidase; glutathione S-transferases and bile binding proteins including ileal bile acid binding proteins and liver fatty acid-binding proteins, prostaglandin receptors including PPARg, prostacyclin receptors including PTGIR and thromboxane receptors such as TXA2; L-ascorbate binding protein including L-ascorbate oxidase; galactose binding protein including galactose oxidase, xanthine oxidase, xanthine dehydrogenase, phosphoribosyltransferase, xanthine binding RNAs, catecholamine regulated protein (CRP40), catecholamine binding proteins, adrenergic receptors (alpha and beta), epinephrine receptor, norepinephrine receptor; nucleotide binding proteins such as G proteins and ATP binding proteins respectively. Because a small molecule is selected to be a tracer on the basis that it will interact specifically with a corresponding biomacromolecule, the biomacromolecule does not have to be added to an industrial fluid conducting and containment system. This is advantageous because it is not exposed to the damaging harsh conditions typically present in such systems. The tracer, on the other hand, is robust under such conditions. Thus, the detection of the tracer can be conducted under conditions that are optimised to be suitable for correct functioning of the biomacromolecule. Furthermore, because these tracer biomacromolecule pairs all have the feature that they associate specifically in nature, he user may be certain that the signal detected on addition of a biomacromolecule to the sample containing the tracer is due to the presence of the tracer alone.

Optionally, the tracer may be associated with at least one treatment substance, the treatment substance being used for maintaining efficient flow within a fluid system. The treatment substance may be selected from; scale inhibitors, corrosion inhibitors, hydrate inhibitors, wax inhibitors, anti-fouling agents, asphaltene inhibitors, hydrogen sulphide inhibitors, pH stabilisers, flow additives, anti-foaming agents, hydrogen sulfide scavengers, detergents and demulsifiers or a microorganism. This feature enables the concurrent use of the tracers both as tracers for fluid flow and also to analyse distribution of treatment substances or microbes within the system. This feature additionally provides the possibility of assessing the movement of such treatment substances, as measured using the tracer, relative to the fluid flow, measured using the free tracer.

The signal may be detectable by a fluorescence detector, luminescence detector, Raman detector, optical microscope, CCD camera, photographic film, fibre-optic device, photometric detector, MEMS (micro-electro-mechanical-systems) device, single photon detector, spectrophotometer, chromatography system or by eye. The person skilled in the art will understand that the method of detection will be selected on the basis of the type of tracer-biomacromolecule pair used for the treatment chemical.

Preferably, the tracer will be detectable at a concentration of at least 1 ppb when in the presence of a biomacromolecule. Such a low concentration allows the tracer to be detected even at low levels. Therefore, the concentration can be kept as low as is necessary to reduce the amount of tracer that will be wasted.

The tracers described hereinabove are of particular use within fluid conducting and containment systems that require high flow efficiency in order to achieve high productivity.

Such systems include oil and gas reservoirs and their associated infrastructure (wells, pipelines, separation facilities etc), petrochemical processing facilities, refineries, paper manufacture, mining, cooling towers and boilers, water treatment facilities and water systems e.g. lakes, reservoirs, rivers, and geothermal fields. The advantages of this method for these particular systems are numerous. The detectable signal is specifically indicative of the presence of the tracer because the signal is only produced if the biomacromolecule has been added and the tracer is present. The reagents are cheap and easy to store on off-shore or remote locations, such as oil fields or drilling rigs. The tracers can be monitored close to the system, preventing time delays in detecting changes in the flow of fluid within the system that might occur if the samples had to be transported before testing. The tracers are especially useful for these systems because the common problems of signal interference due to contaminants such as treatment chemicals, oil etc are overcome using latently detectable molecules, because a simple background signal subtraction ensures that any signal is attributable to the presence of the composition.

According to a second aspect of the present invention, there is provided a method of monitoring the flow of fluid through a system for the conduction and containment of fluid comprising adding a predetermined amount of at least one tracer according to claim 1 at a first location in the system, adding a biomacromolecule as hereinabove described to the fluid in at least one second location in said system, said second location being downstream of the first location, wherein the predetermined amount of the detectable tracer at the first location is sufficient for the concentration of the detectable tracer at the second location to be above its detection limit of 1 ppb, the concentration of the biomacromolecule being sufficient to produce a detectable change in the fluid due to a specific interaction of the tracer with the biomacromolecule; detecting the change in the fluid, analysing the measured detectable change to determine the concentration of the tracer at the second location, and using the data obtained by detecting, measuring and analysing the change to assess flow characteristics of the fluid within the system.

This embodiment of the invention advantageously provides a convenient, cost-effective method of monitoring fluid flow in a fluid system, which addresses the problems of strong background or interferences in a sample such as autofluorescence in an oil solution. This is because the tracer is latently detectable, and therefore the signal emitted by the fluid could be measured before and after the addition of the biomacromolecule. The signal measured before addition would be subtracted from the signal measured after addition. The difference between the signals would then be attributed to the interaction between the tracer and the biomacromolecule. This sampling and testing method can be performed on site, reducing or replacing the need for expensive transportation of samples, expensive specialist equipment or other complicated and time-consuming practices. The tracer-biomacromolecule pairs used in this method all have the feature that they associate specifically, so that there is reduced possibility that any non-specific interaction may occur which could lead to a false-positive signal. The tracer and its distribution in a system may therefore be detected and analysed accurately, enabling a rigorous assessment of the fluid flow in the system.

Optionally, a sample may be taken from the second location so that the monitoring is done outside the system. This will be useful, for example, where the biomacromolecule or any other molecules used to generate a signal due to the presence of the composition cannot be added directly to the fluid in the system. In such a case, the sample could be removed completely from the system or diverted away from the main system so that the conditions can be optimised for functioning of the biomacromolecule.

The sample taken may be treated to improve detection of the signal. This may involve concentration of the sample, bleaching to remove background fluorescence, filtration to remove impurities or immobilisation or extraction. This may improve the detectability of the signal resulting from the interaction between the tracer and the associated biomacromolecule. Such treatment could take place before or after the addition of the biomacromolecule. This may be especially useful where there is a high background fluorescence, other interfering chemicals, or where the signal from the label itself is known to be difficult to detect.

The detectable change may be an optical signal. The signal may be fluorescent, luminescent signal or a colour change, or may be a spectroscopic change such as an altered raman signature. Where the signal is luminescent, spectroscopic or a colour change, autofluorescence from the sample (for example from oil or other contaminants), would not create background noise during measurement of the signal due to the composition in the sample.

The detectable change may be a chemical signal, such as the production of a chemical. Chemical changes are very easy to detect, especially where the chemical would not be expected to be present in a fluid unless the interaction has taken place.

The method may further comprise a step of adding a second molecule before detecting the change in the fluid or sample. This step will be useful where the interaction of the biomacromolecule with the tracer leads to production of a chemical. The second detection molecule can be used to convert the chemical into a fluorescent or coloured product for detection. The second molecule could interact with the chemical product and produce a signal. Detection of a particular chemical product in a sample in this way is a very simple and convenient method for assessing whether the interaction has taken place. As the interaction can only take place when both the biomacromolecule and the tracer is present, the presence and/or concentration of the tracer will be easy to determine. The use of a second molecule may also be useful, for example, where it is required in order to develop an optical signal resulting from the interaction between a label and a biomacromolecule.

The chemical may be hydrogen peroxide. The second molecule may be 10-acetyl-3,7-dihydroxyphenoxazine (ADHP, Amplex® Red) which, in the presence of peroxidase, generates the highly fluorescent product resorufin. The fluorescence emitted from the sample due to the presence of this highly fluorescent product may then be detected and attributed to the presence of the composition. Any background fluorescence may be measured before addition of the second molecule and enzymes, and this measurement subtracted from the measurement of fluorescence after addition of the second molecule and enzyme.

The second molecule may alternatively be Phenol Red, which would be added with peroxidase. The Phenol Red would undergo a change in absorbance at 610 nm in the presence of the hydrogen peroxide and peroxidase. A colorimetric assay such as this is particularly useful where the sample fluid is colourless, or where the colour produced during the assay is different to that of the sample fluid. The colour signal is indicative of the presence of the treatment composition in the sample.

The second molecule may alternatively be ferrous ions which are oxidised to ferric ions in the presence of hydrogen peroxide and which interact with the indicator dye xylenol orange to produce a purple coloured complex measureable at 560-590 nm. Optionally, sorbitol may be included in the reaction mixture to amplify the color intensity.

The second molecule may be a cyclic diacyl hydrazide such as luminol. Such molecules are converted to an excited intermediate dianon in the presence of hydrogen peroxide and horseradish peroxidase. This dianion emits light on return to its ground state. Phenols can be used to enhance the reaction up to 1000-fold.

Multiple tracers may be monitored, each being detectable using different signals. This allows the user to detect the different tracers using different signals, conveniently and in one assay. This is a simple and efficient method of assessing the concentration of many tracers within a system. This may be especially useful where the relative proportions of tracers in a commingled fluid containing fluids from different pipes or sources needs to be known. If these different substances are assessed at different times, using different experiments, inaccuracies and time delays may occur in this assessment so that the relative proportions cannot be calculated.

The optical signal is preferably detectable by a fluorescence detector, luminescence detector, Raman detector, optical microscope, CCD camera, photographic film, fibre-optic device, photometric detector, MEMS device, single photon detector, spectrophotometer, chromatography system or by eye.

Optionally, the monitoring method can be performed off line. An off-line method allows the user to take a sample from a fluid conducting and containment system, and analyse it at a later stage. Such a system is useful where a sample has been taken from an off-shore oil rig, and the oil rig has become too hazardous for carrying out assessment of the sample. In such cases, the equipment and personnel for analysis of the sample may be located far from the location at which the sample is taken.

Optionally, the monitoring method can be performed inline. An in-line method could involve the use of a loop diverting a small but representative sample volume of fluid from the main flow. The biomacromolecule could be injected into the loop, the sample could then feed into a flow cell and the signal detected by, for example, a snapshot imager or by fluorescence reading. An in-line method would advantageously provide the user with real-time data reflecting the composition of the multiphase sample. In line methods of analysis are preferable to other methods because they provide the means for real-time monitoring of samples that are as representative as possible of the situation in the fluid conducting and containment system. An in line method allows frequent, real-time monitoring as samples do not have to be collected from the bulk flow of the system. In addition, the fluid conducting and containment system does not need to be shut down in order to conduct the monitoring tests.

Optionally, the monitoring method may be performed atline. An at-line method allows the user to remove a sample from the fluid conducting and containment system and analyse it on site, close to the fluid conducting and containment system. This monitoring method is not real time but is rapid, and all of the equipment is portable and may be automated, making this method of testing suitable for offshore use. It may be useful to employ such a method when a biomacromolecule cannot be added to an inline loop in the case that conditions are detrimental to the functionality of the biomacromolecule. In addition, the fluid conducting and containment system does not need to be shut down in order to conduct the monitoring tests.

Optionally, the monitoring method may be performed online. An online method may be used as part of an automated monitoring process, which feeds directly into a computerised monitoring system for monitoring offsite. For example, an online monitoring method may incorporate an automated in-line loop from the main fluid conduction and containment system, information from the in-line loop being recorded directly to the operator's computer system so that technicians at a different location may review it. This method advantageously allows data to be recorded in real time, but the personnel required to analyse the data would not need to be on-site. Online monitoring has a number of advantages; no manual handling of the sample is required, there is an immediate response (<1 second) and the result can be correlated to a recognised standard reference method. This monitoring method could be used to provide information where the biomacromolecule is added directly to the flow of fluid, and the signal resulting from an interaction with the label is recorded by an online detector. In addition, the fluid conducting and containment system does not need to be shut down in order to conduct the monitoring tests.

The method may use a tracer that is associated with a treatment substance, as hereinabove described, the treatment substance being used for maintaining efficient flow within a fluid conduction and containment system. The treatment substance may be selected from; scale inhibitors, corrosion inhibitors, hydrate inhibitors, wax inhibitors, anti-fouling agents, asphaltene inhibitors, hydrogen sulphide inhibitors, pH stabilisers, flow additives, anti-foaming agents, hydrogen sulfide scavengers, detergents and demulsifiers or a microorganism. This feature enables the concurrent use of the tracers both as tracers for fluid flow and also to analyse distribution of treatment substances or microbes within the fluid conduction and containment system. This feature additionally provides the possibility of assessing the movement of such treatment substances, as measured using the tracer, relative to the fluid flow, measured using the free tracer.

The method, where a tracer associated with a treatment substance has been used, may further include the step of using the data to inform administration of the at least one treatment substance into the fluid conduction and containment system in order to maintain effective concentrations of said treatment substances. This feature is particularly useful because it provides a method of reducing waste of treatment substances (as treatment substance will only be added when necessary), of maintaining effective concentrations of treatment compounds and allows early detection and implementation of preventative action to minimise risks of production losses. The method can also be advantageously used to provide quantitative evidence of treatment substance usage, with advantages for monitoring of environmental impact of treatment substances.

The method of monitoring described hereinabove is of particular use within fluid conducting and containment systems that require high flow efficiency in order to achieve high productivity.

Such systems may include oil and gas reservoirs and their associated infrastructure (wells, pipelines, separation facilities etc), petrochemical processing facilities, refineries, paper manufacture, mining, cooling towers and boilers, water treatment facilities and water systems e.g. lakes, reservoirs, rivers, and geothermal fields. The advantage of this method for these particular fluid conduction and containment systems is that the method is highly specific to the tracer, the signal is only produced on addition of the biomacromolecule, the reagents are cheap and easy to store on off-shore or remote locations, and the method can be performed close to the fluid conducting and containment system, preventing time delays in detecting changes in the flow of fluid within the fluid conducting and containment system. The method is especially useful for these industries for a number of reasons relating to problems with interference due to contaminants such as treatment chemicals, oil etc. Therefore, a simple background signal subtraction will allow detection of the treatment chemical in question.

According to a third aspect of the invention, there is provided a kit for use in monitoring the flow of fluid through a system for conduction and containment of fluid, comprising; a tracer as hereinabove described; and a biomacromolecule selected accordingly to the tracer included in the kit. The kit may further include means for taking a sample from said system.

The kit may further include a second detection molecule. This would be convenient if the interaction between the tracer and the biomacromolecule leads to a chemical change in the sample. The second detection molecule could then interact with the chemical product and produce a detectable signal.

The kit may also include an optical detector selected from a fluorescence detector, luminescence detector, Raman detector, optical microscope, CCD camera, photographic film, fibre-optic device, photometric detector, MEMS device, single photon detector, spectrophotometer or chromatography system.

A number of embodiments of the invention will now be described, reference being made to examples, experimental data and accompanying figures in which:—

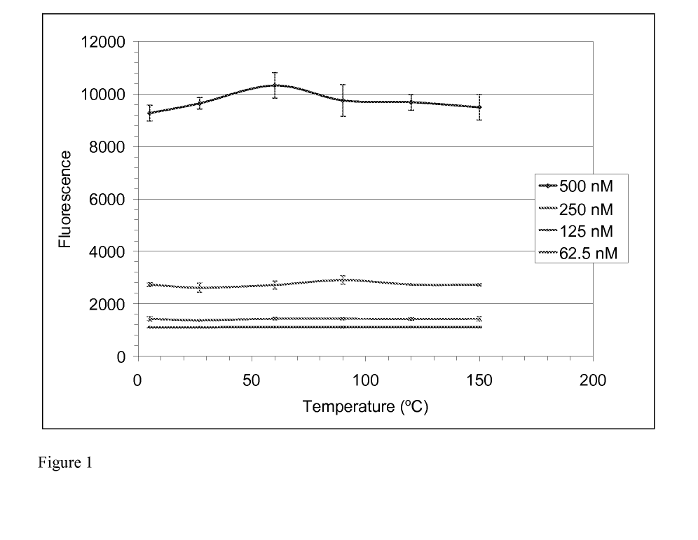

FIG. 1 is a graph showing the detectability of d-biotin at increasing temperatures and concentrations;

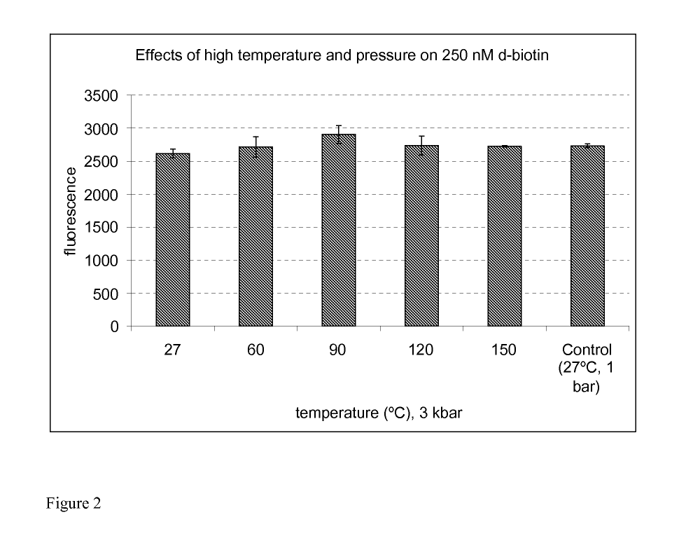

FIG. 2 is a graph showing the detectability of 250 nM d-biotin at high temperatures and pressures;

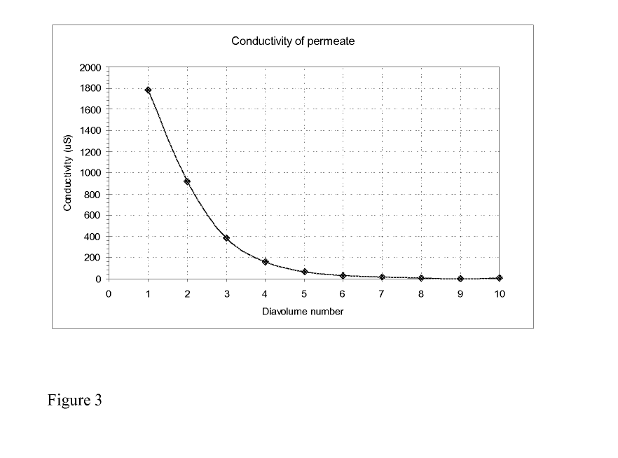

FIG. 3 is a graph showing decrease in conductivity of a solution of biotin as the biotin is taken up by a reagent;

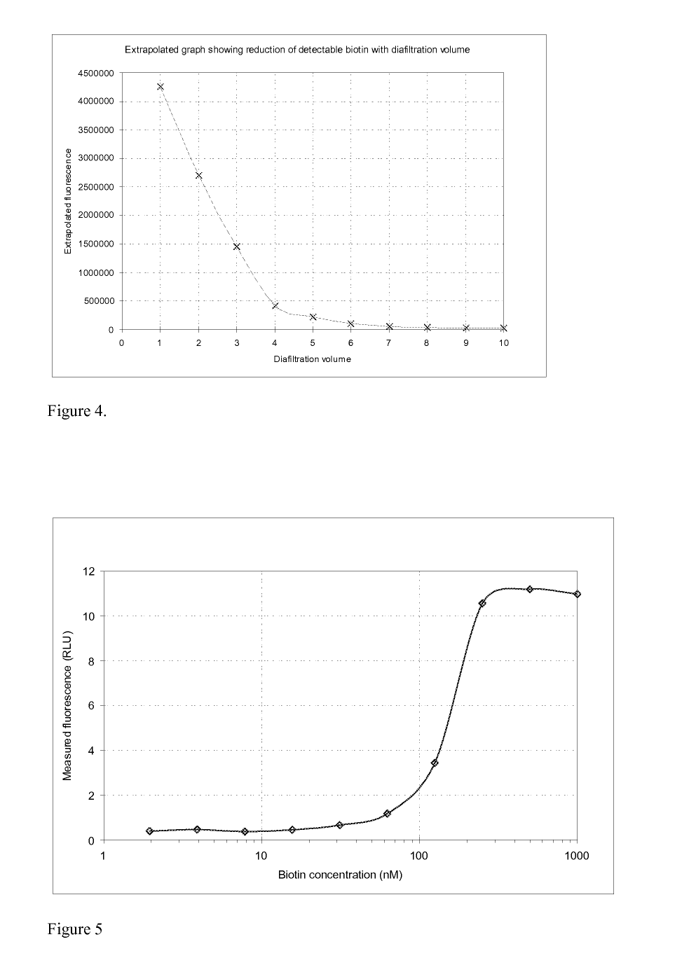

FIG. 4 is a graph showing that as the biotin is taken up by a reagent, the fluorescence of a solution decreases accordingly;

FIG. 5 is a graph showing the partitioning of biotin in various solutions;

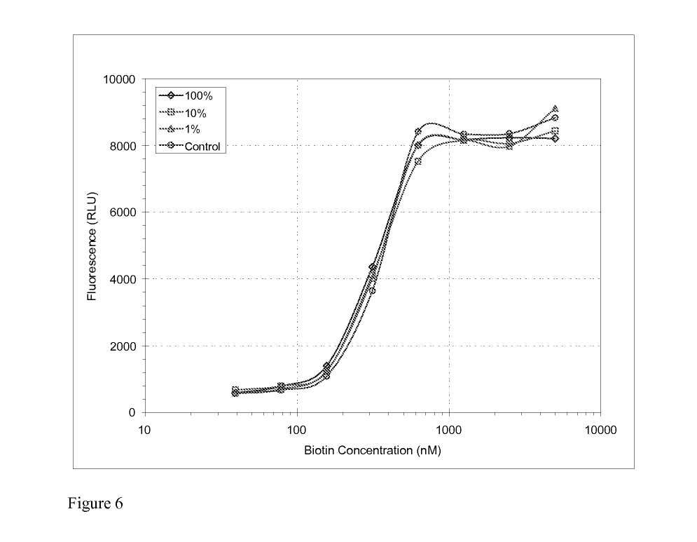

FIG. 6 is a graph showing the limit of detection (LOD) of biotin-tagged scale inhibitor;

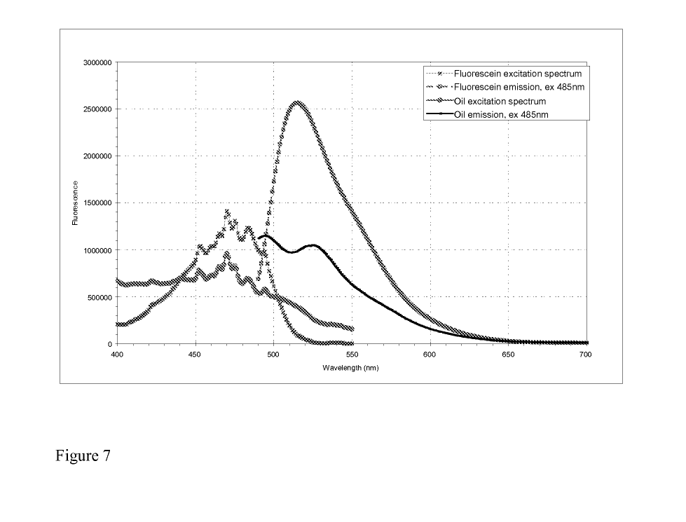

FIG. 7 is a graph showing excitation and emission spectra of 0.1 mg/cm3 fluorescein and the oil fraction from Miller field produced fluids, diluted to 0.1% in petroleum ether (non-fluorescent);

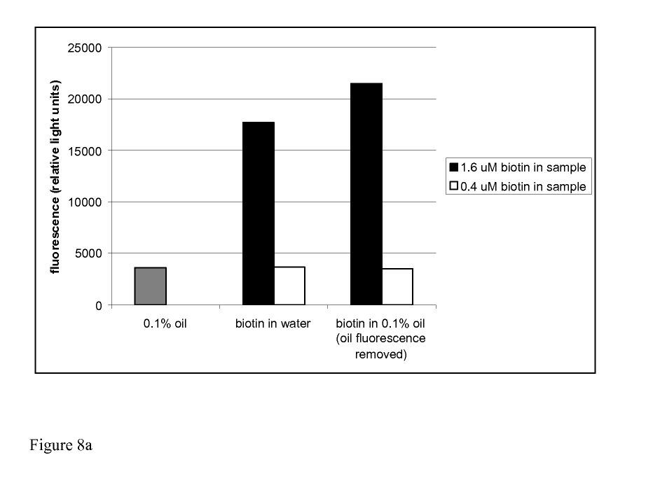

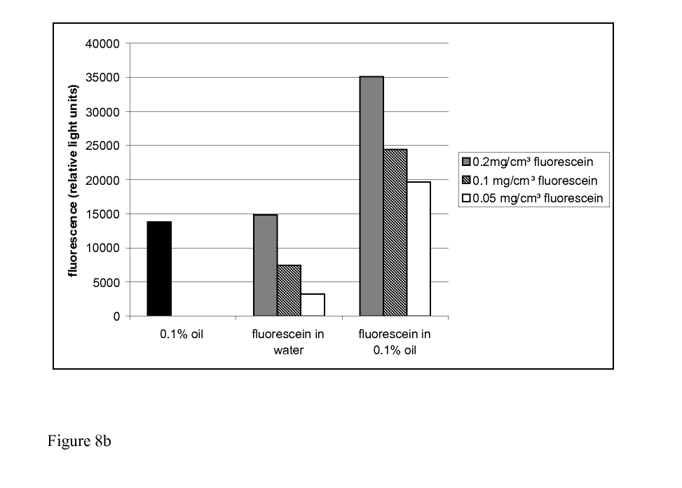

FIG. 8a is a graph showing the fluorescence detected from various concentrations of biotin in deionised water or 0.1% oil;

FIG. 8b is a graph showing the fluorescence of various concentrations of fluorescein in deionised water or 0.1% oil;

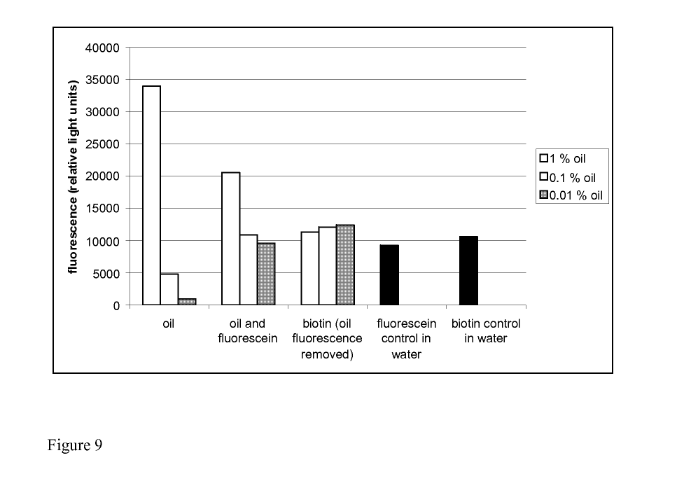

FIG. 9 is a graph showing the fluorescence of tracer (either 0.8 μM biotin or 0.1 mg/cm3 fluorescein) when mixed with 1%, 0.1%, 0.01% of oil;

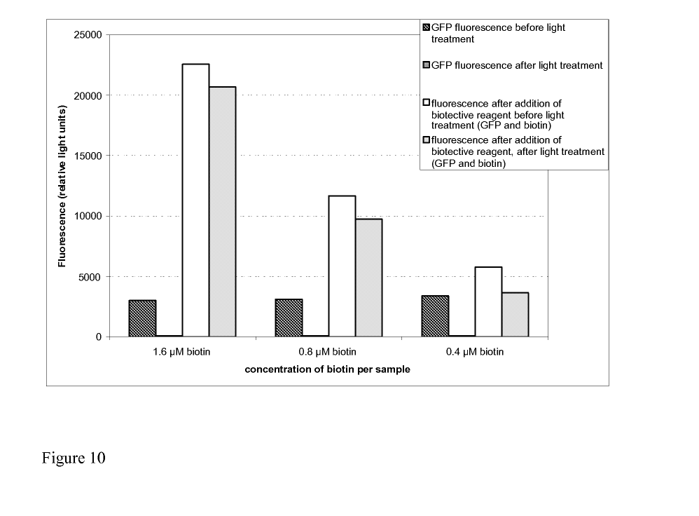

FIG. 10 is a graph showing the fluorescence of a solution of GFP (0.1 mg/ml Renilla reniformis protein, 80%, in water) with added biotin, (a) no treatment (b) heat treated (samples were heated to 100° C. for 1 hour in an oven);

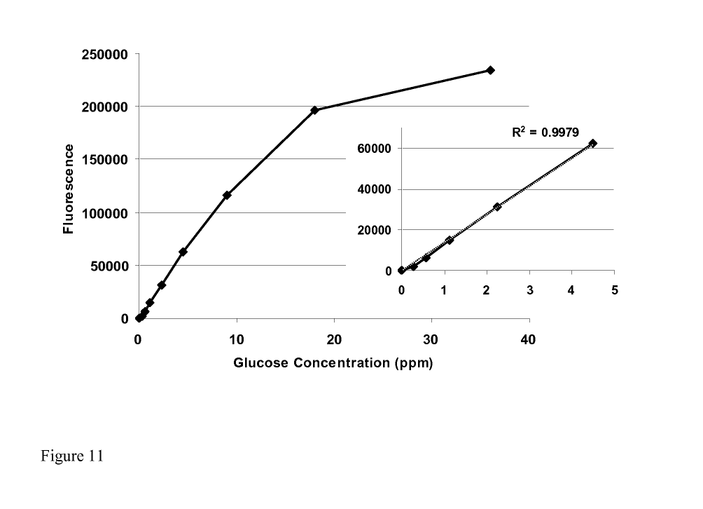

FIG. 11 is a graph showing a calibration curve for a range of glucose concentrations. The inset shows a linear fit (R²=0.9979) of the data points for concentrations 0-4.5 ppm;

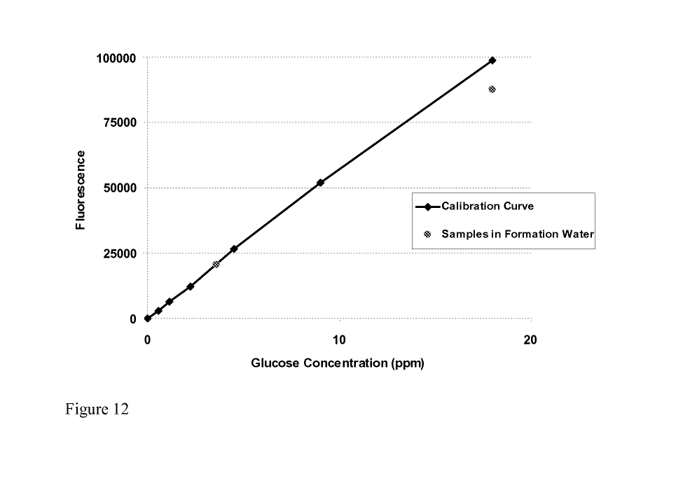

FIG. 12 is a graph showing a comparison between glucose samples prepared in synthetic formation water and the calibration curve which was generated using aqueous glucose samples;

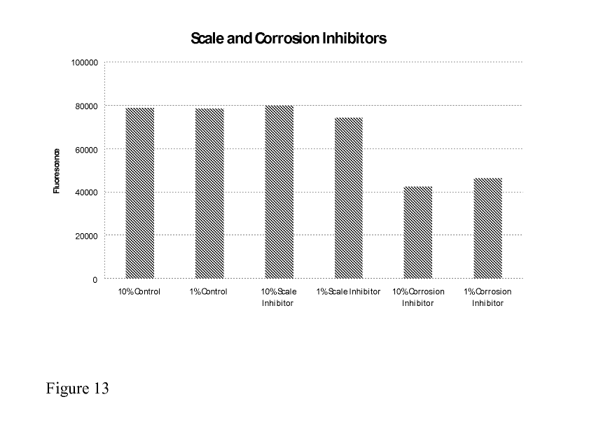

FIG. 13 is a graph showing the effects of scale inhibitor 8017C and corrosion inhibitor EC1440A on the concentration of glucose detected. The graph shows the average of duplicate samples;

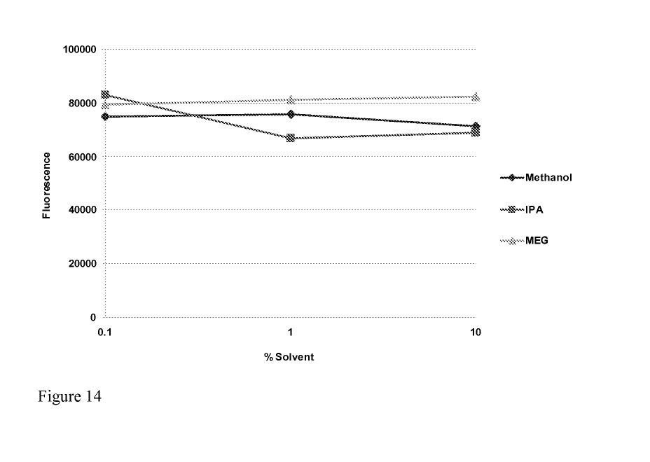

FIG. 14 is a graph showing results from the glucose assay when carried out in the presence of various concentrations of methanol, IPA and MEG. An aqueous glucose control sample with no added solvent gave a fluorescence reading of 80,227;

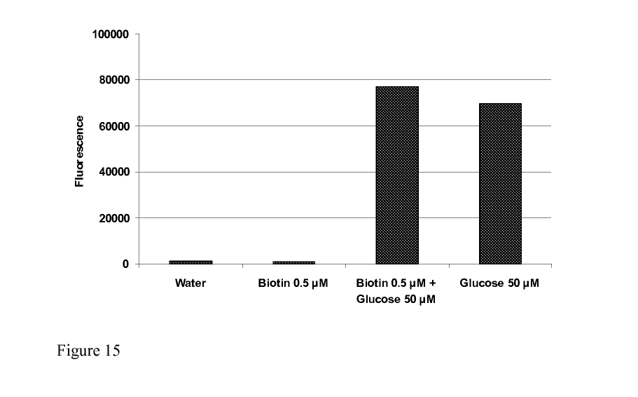

FIG. 15 is a graph showing the detectability of glucose in the presence of biotin;

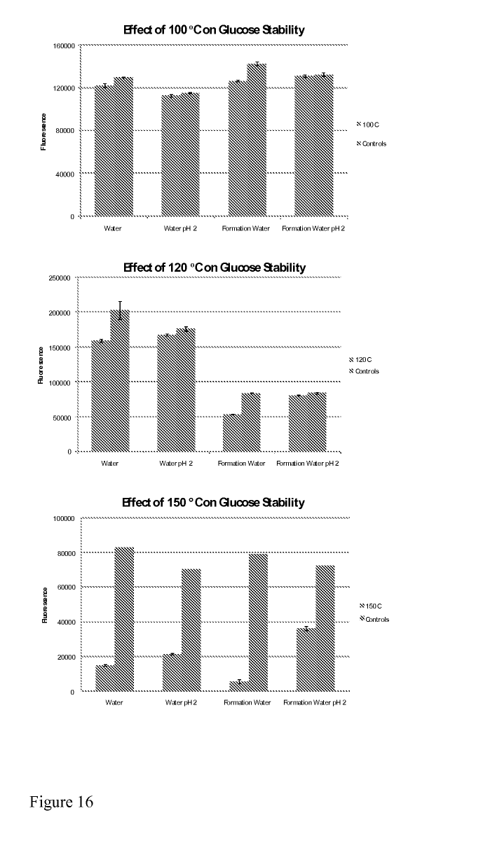

FIG. 16 is a set of graphs showing the stability of glucose at 100, 120 and 150° C. in water and formation water at neutral and low pH;

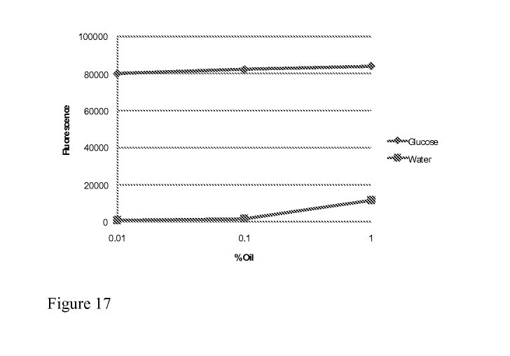

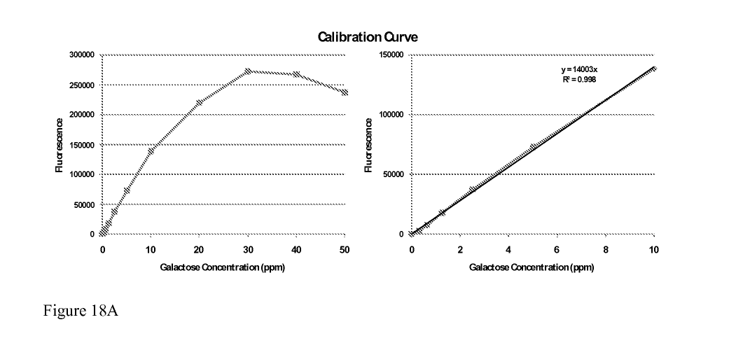

FIG. 17 is a graph showing the effect of crude oil on the glucose assay. Control (water plus glucose) fluorescence value 78,492;

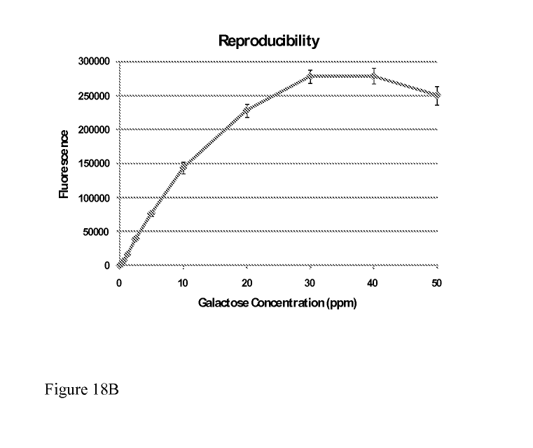

FIG. 18A is a set of two graphs showing a calibration curve for galactose concentrations of 50, 40, 30, 20, 10, 5, 2.5, 1.25, 0.625, 0.3125 and 0 ppm, and also a linear fit (R²=0.998) of the data points for concentrations 0-10 ppm is shown;

FIG. 18B is a graph showing the results of analysis of the calibration curve samples (0-50 ppm) on three different days with fresh assay reagents prepared each day. The error bars represent 95% confidence intervals;

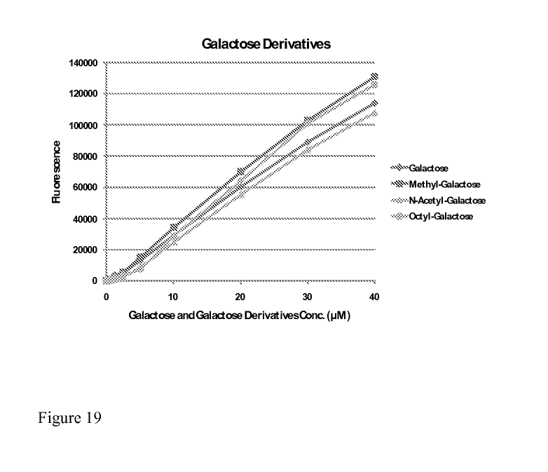

FIG. 19 is a graph showing a range of concentrations of galactose derivatives were analysed and the fluorescence values compared to those for galactose;

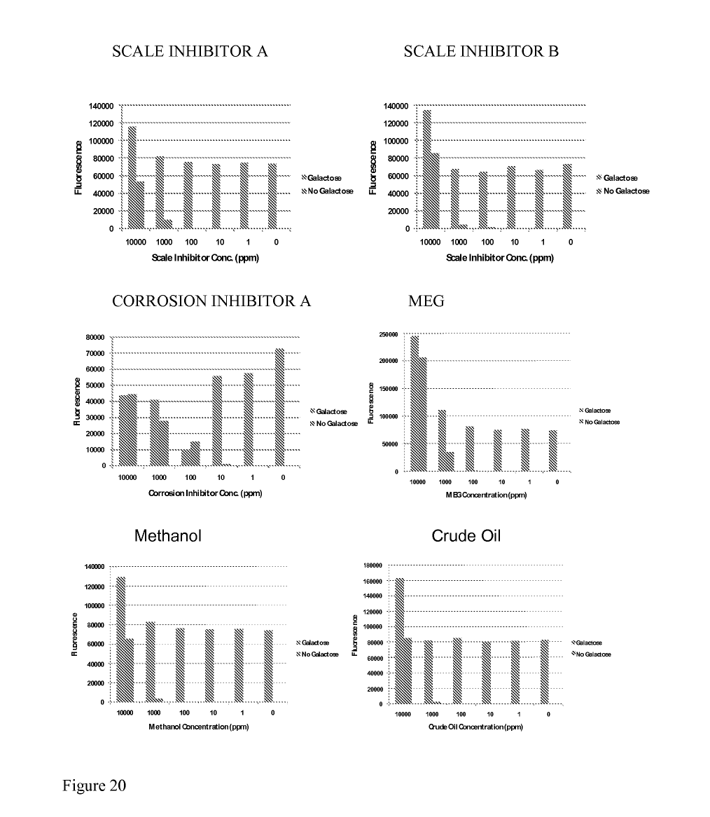

FIG. 20 is a set of graphs showing the effect of various interferences on the galactose assay;

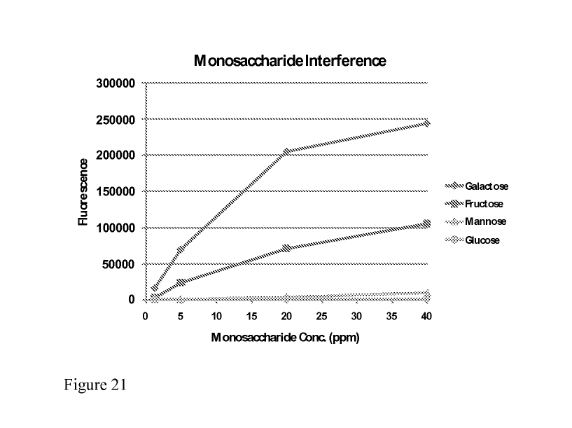

FIG. 21 is a graph showing the results of an assay on various concentrations of fructose, mannose and glucose to determine whether other monosaccharides could be oxidised by galactose oxidase;

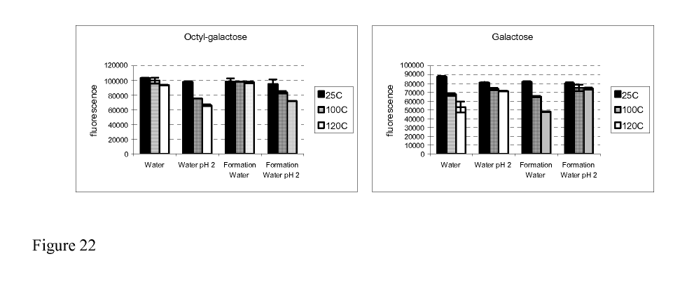

FIG. 22 is a graph showing the stability of galactose and octyl-β-galactose at 25, 100 and 120° C. in water and formation water at pH 6-7 and pH 2. The error bars represent 95% confidence intervals from triplicate samples;

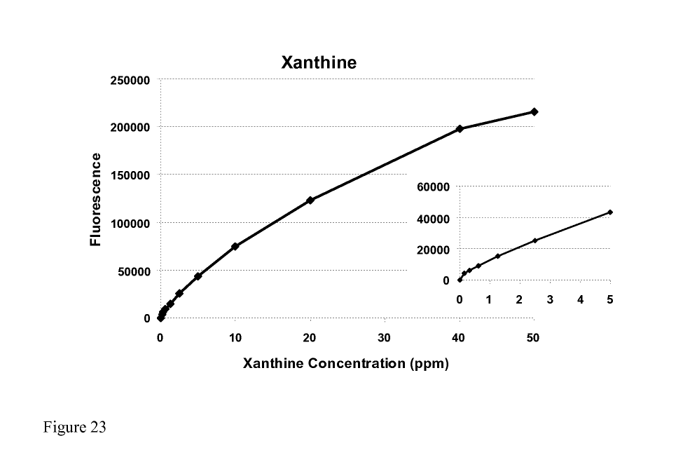

FIG. 23 is a graph showing a calibration curve for xanthine concentrations of 50, 40, 20, 10, 5, 2.5, 1.25, 0.625, 0.3125, 0.15625 and 0 ppm. The inset has zoomed in on the lower concentration region;

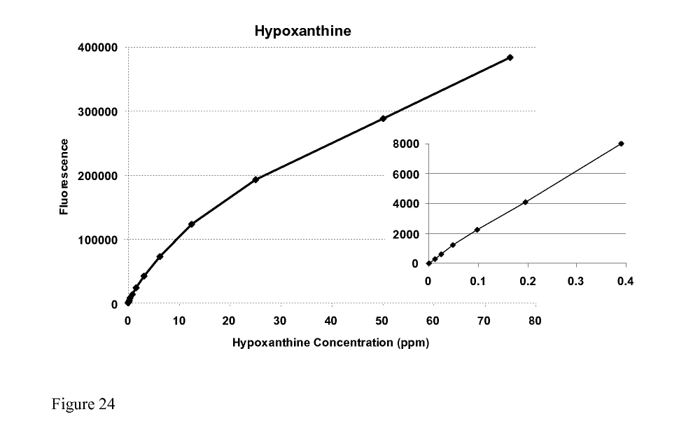

FIG. 24 is a graph showing a calibration curve for hypoxanthine concentrations of 75, 50, 25, 12.5, 6.25, 3.125, 1.5625, 0.78125, 0.3906, 0.1953, 0.0977, 0.0488, 0.0244, 0.0122 and 0 ppm. The inset has zoomed in on the lower concentration region;

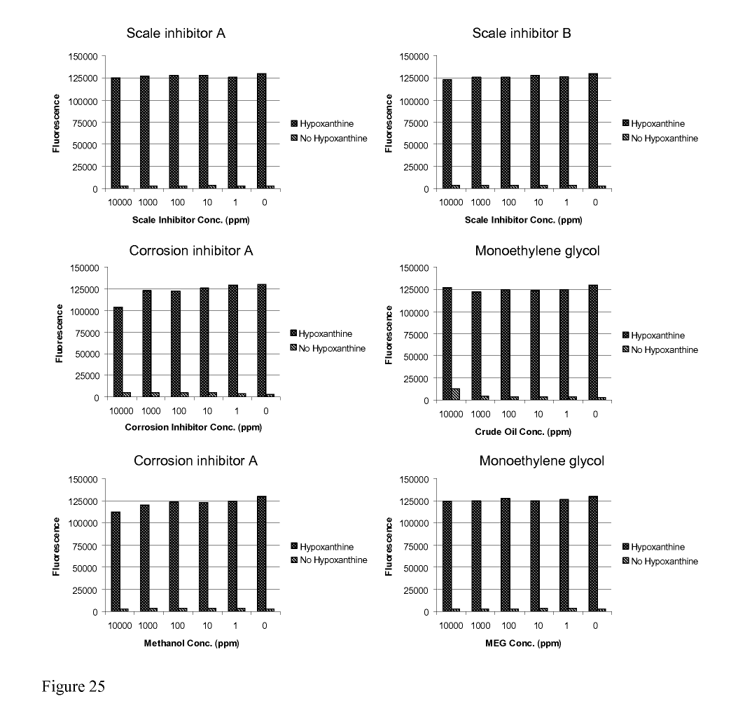

FIG. 25 is a set of graphs showing the effect of various interferences on the xanthine and hypoxanthine assay;

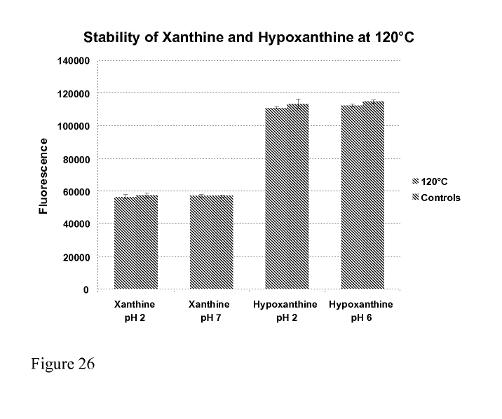

FIG. 26 is a graph showing the stability of xanthine and hypoxanthine at 25 and 120° C. at pH 6-7 and pH 2. The error bars represent 95% confidence intervals from triplicate samples;

### EXAMPLE 1

### Resistance of d-Biotin to High Temperatures and Pressures

Many biomacromolecules, such as streptavidin, act as part of complexes in nature, with recognition sites for specific small molecules (such as biotin, in the case of streptavidin) that influence binding and function of the biomacromolecule. Indeed, one of the most common ways in which a molecule may exert its effect in a plant or animal is through a specific association with another molecule, the association leading to a cascade of such molecular signalling events. Such a biomacromolecule-small molecule complex is known as a molecular signalling complex. The binding of a small molecule to its recognition site in the biomacromolecule may lead to displacement of another small molecule, production of a molecule or to a conformational, light or colour change in a sample. The displaced small molecule, the produced molecule or the conformational change can be detected. By detecting the displaced molecule, the quantity of the target small molecule that was bound to the recognition site can be detected. Similarly, the emitted light, produced molecule or colour change can be calibrated to the amount of the small molecule that is bound to the recognition site. Such a method is frequently used within the context of biological, biomedical and biochemical fields of application.

In particular, biotin (Formula: C₁₀H₁₆N₂O₃S), also known as vitamin H or B₇, is a good example of a useful tracer or marker. It is small, commercially available in large quantities and there are a number of functionalised versions available e.g. biotin ethylene diamine, biotin cadaverine and biotin hydrazide which have amine groups that can be used to bind to carboxylic acid-containing chemicals e.g. some scale inhibitors. Biotin is a prosthetic group found on only a few protein species (Ann N.Y. Acad. Sci 447:1-441, Dakshinamurti and Bhagavan, Eds. (1985)). In nature, biotin has roles in the catalysis of essential metabolic reactions to synthesise fatty acids, in gluconeogenesis and to metabolise leucine. One of the most important features of biotin is its very strong binding to streptavidin, avidin, neutravidin and captavidin proteins. Binding of biotin to avidin has a dissociation constant K_{d} in the order of 10⁻¹⁵ mol/L (Bonjour, 1977; Green 1975; and Roth, 1985). Harsh conditions are required to break the biotin-streptavidin bond i.e. high temperatures, extremes of pH and denaturing conditions.

This strong association has lead to much research into how molecules bind. The strong bond also accounts for the use of biotin in many biological applications. For example, biotin may be linked to a molecule of interest for biochemical assays e.g. proteins, enzymes, peptides, oligosaccharides and lipids. If avidin/streptavidin/neutravidin/captavidin are added to the mixture then they will bind to the biotin. This can allow capture of the biotin tagged material. Such an approach is typically used in, for example, enzyme-linked immunosorbant assays (ELISA), a biochemical technique used mainly in immunology to detect the presence of an antibody or an antigen in a sample; enzyme-linked immunosorbent spot (ELISPOT), a common method for monitoring immune responses in humans and animals; and affinity chromatography, a method for separating biochemical mixtures (also may be used in protein purification). Application of biotin has been limited to tools for microbiology, biochemistry and medical science. There are no examples of biotin being used to monitor the flow of fluids in fluid conducting and containment systems according to the invention.

Biomacromolecules are highly sensitive to their surroundings. For example, high or low temperatures and/or solutions of high or low pH can often denature proteins, destroying their ability to bind a small molecule and affecting their functionality. As a result, amino acid derivatives such as polypeptides are not ideally suited for introduction into fluid conducting and containing systems, either attached to oil or water treatment substances or as free moieties. In addition, biomacromolecules are large, and therefore could potentially have a major impact on the fluid conducting and containment system being investigated, in particular if they are prone to coagulation.

The resistance of d-biotin to high temperatures and pressures was investigated. A dilution series of d-biotin was made and exposed to increasing temperatures and 4 kbar pressure in the presence of the aqueous phase of produced fluids (from Miller, Foinaven and Schiehallion fields). The ability of heat and pressure-treated and -untreated samples to bind streptavidin determined using Biotective green (Invitrogen) was investigated. In this detection method fluorescein (a fluorescent dye) is attached to streptavidin but can only fluoresce when biotin binds, and a quencher is removed. No loss of fluorescence was detected due to temperature and pressure, even at 150° C. (FIG. 1).

The vitamin was then diluted in formation water (synthetic, based on formation water from the Forties Field in the North Sea) to 250 nM and 300 ul was exposed to 15 minutes of 3 kbar pressure at 28, 60, 90, 120 or 150° C. The ability of heat and pressure-treated and -untreated samples to bind streptavidin was determined using Biotective green (Invitrogen). In this detection method fluorescein (a fluorescent dye) is attached to streptavidin but can only fluoresce when biotin binds, and a quencher is removed. Results indicate that there was no obvious drop in fluorescence even after exposure to 150° C., 3 kbar, for 15 minutes. Representative results from 250 nM solutions are shown in FIG. 2. Biotin appears sufficiently robust to high temperatures and pressures to be used as a tag.

### EXAMPLE 2

### Detectability of Free Biotin in Solution

The biotective green assay (Invitrogen) was used to detect the concentration of biotin in samples. It was used according to manufacturers instructions. A standard curve was first generated to enable quantification of the amount of biotin in each sample. The conductivity and concentration of biotin in permeate was determined. As free biotin (that has not been coupled to scale inhibitor) is successfully removed from the solution of tagged scale inhibitor and free biotin, the conductivity and fluorescence of the samples decreases (see FIGS. 3 and 4 respectively). This data suggests that biotin in solution can be detected simply by addition of an associated biomacromolecule, and that changes in concentration may also be detected.

In the event that, when the tracers are used in a fluid conducting and containment system, there is no detectable change in the sample taken (i.e. the amount of detectable tracer is below the detection limit of 1 ppb), the amount of detectable tracer that is added to the fluid at the first location would be increased until a detectable change is measured in a further sample removed at the second location. The skilled person would understand that the predetermined concentration of detectable tracer at the first location will be dependent upon factors such as the rate of any degradation of the detectable tracer in the fluid under the conditions encountered in the conducting and containment system or the rate of any loss of the detectable tracer, for example, owing to interactions of the tracer with components of the fluid or to absorption of the tracer onto the internal surfaces of the fluid conducting and containment system. Preferably, the half-life of the detectable tracer in the fluid under the conditions in the conducting and containment system is determined and the amount of detectable tracer that is added at the first location is adjusted to ensure that a detectable change is produced in the sample at the second location. The person skilled in the art will understand that the predetermined amount will be dependent upon both the half-life of the detectable tracer and the time taken for the fluid to flow from the first to the second location.

### EXAMPLE 3

### Detection of Biotin

To be useful, tracers need to be detectable at very low levels, ideally below 1 ppm. A dilution series of D-biotin was prepared in deionised water and a modified protocol of the Biotective Green assay, which utilized larger volumes of reagent in a cuvette format and the PicoFluor fluorometer (TurnerBiosystems) was used to determine the limit of detection of D-biotin. Results indicate that limits of detection to 20 nM (5 ppb) are possible, FIG. 5.

### EXAMPLE 4

### Toxicity of Biotin

Before offshore use chemicals must be tested to provide information for registration (UK). The toxicity of biotin was assessed using Marine Unicellular algae Skeletonema costatum, ISO DP 10253 (1998) Standard Method. The biodegradation of biotin was assessed with a 28 day seawater test, OECD 306. Results indicate that D-biotin exhibited no toxic effect at 4462.48 mg/L to S. costatum. D-biotin degraded by 14% over 28 days and showed an inhibition of 42% to seawater bacteria.

### EXAMPLE 5

### Robustness of Biotin in Contact with Treatment Chemicals Used in the Oil Industry

To be useful tracers need to be robust in the contact of treatment chemicals that may also be present e.g. scale inhibitors. 50 mM D-biotin was incubated (1:10) with a corrosion inhibitor (TROS787c, Clariant) for 2 hours. The samples were then diluted 1:5000 in formation water (>1M NaCl) and the level of detectable biotin assayed using the Biotective Green assay (Invitrogen). No significant affect of corrosion inhibitor on biotin was observed (FIG. 6).

### EXAMPLE 6

### Data Showing the Advantage of Using Latently Detectable Tags and Impact of Background Interferences

Many fluids in containment systems interfere with the detection of tracers. Fluids may be coloured, or have autofluorescence, such as oil solutions. Where the tracer is fluorescent it will be difficult to quantify the amount present if there is interfering autofluorescence from the sample. However, if the tracer is latently detectable then the autofluorescence from the sample can first be assessed, then the fluorescence directly attributed to the tracer determined. This is the case in FIGS. 8 and 9, where quantification of a latently detectable biotin tracer in oil is compared with fluorescein, a commonly used fluorescent tracer.

In both experiments, fluorescence from samples was detected at 485 nm excitation and 535 nm emission. Oil is also known to fluoresce at this excitation and with overlapping emission, see spectra in FIG. 7, which shows excitation and emission spectra of 0.1 mg/cm3 fluorescein and the oil fraction from Miller field produced fluids, diluted to 0.1% in petroleum ether (non-fluorescent). For fluorescein-containing solutions samples were measured directly and for solutions containing latently detectable biotin, fluorescence from the oil solution was first determined at 485/535 nm (excitation/emission) and then Biotective Green assay reagents (Invitrogen) were added to determine fluorescence associated from the biotin, also at 485-535 nm. Measurements were performed in quadruplicate and the average taken.

FIG. 8a shows the fluorescence of various concentrations of biotin in deionised water or 0.1% oil (the oil phase of produced fluid from the Miller field). In FIG. 8b, the fluorescence of various concentrations of fluorescein in deionised water or 0.1% oil (as above) is shown. FIG. 9 shows the fluorescence results of mixing 1%, 0.1% or 0.01% of oil (the oil phase of produced fluid from the Miller field) with one concentration of tracer, either 0.8 μM biotin or 0.1 mg/cm3 fluorescein. Control samples i.e. those without tracer were used to quantify oil autofluorescence.

Both fluorescein and biotin-biotective green cause an increase in fluorescence, beyond that from oil. The difference is that for biotin in the presence of biotective green the background oil fluorescence can be measured prior to addition of biotective green and then subtracted from the signal, providing reliable data for a range of oil and tracer concentrations. For fluorescein that is added directly to the system, it is important to know beforehand the oil concentration in the sample so the end user can determine what fluorescence is from the fluorescein and what is from the oil. In real fluids this concentration may vary and may lead to difficulties in quantification of a directly-fluorescent tracer. However, fluorescein may be useful if added when conjugated to a biomacromolecule (see Example 1)

### EXAMPLE 7

### Data Showing the Advantage of Using Latently Detectable Tags and Pretreating Samples to Minimise Background Interferences

Where a latently detectable tracer is used a sample which contains background interference, such as autofluorescence, can be first treated in some way to minimise autofluorescence. This may be achieved in a number of ways such as addition of chemicals, heat treatment or the bleaching of a sample with autofluorescence. The manner of treatment depends on the sample. This is unlikely to be a viable method if a directly fluorescent tracer is present since these may be adversely affected by the treatment although the latently detectable tracers described here are robust and should remain unaffected.

We took a solution of GFP (0.1 mg/ml Renilla reniformis protein, 80%, in water) and added biotin. The sample has high fluorescence from the GFP. This solution was treated in 2 ways (a) no treatment (b) heat treated (samples were heated to 100° C. for 1 hour in an oven). After treatment fluorescence from the sample was assessed, 485/535 nm excitation/emission, both before and after addition of Biotective Green reagent (Invitrogen) which detects biotin. Results indicated that GFP fluorescence was lost after heating, while the biotin was unaffected, FIG. 10.

Latently detectable tracers are therefore ideal when samples can be treated to minimise inherent fluorescence or background signal. Since such treatment can adversely affect directly detectable fluorescent tracers latently detectable tracers have an advantage.

### EXAMPLE 8

### Limits of Detection of Glucose

The small size and simple structure also make it a good candidate as a tracer. A commercially available Amplex® Red glucose assay was used to determine glucose concentration. An Amplex UltraRed® glucose assay could also be used. Glucose oxidase oxidizes D-glucose to D-gluconolactone producing hydrogen peroxide. In the presence of horseradish peroxidase, H₂O₂ reacts stoichiometrically with Amplex® Red to generate the fluorescent product resorufin which can be detected fluorometrically or spectrophotometrically. The effects of high temperatures, low pH, treatment chemicals, various solvents, high salt concentrations, oil and biotin on detectabilty glucose were investigated.

To determine limits of detection of glucose, initially a calibration curve was generated by analysing glucose solutions prepared by serial dilution (36, 18, 9, 4.5, 2.25, 1.125, 0.5625, 0.28125 and 0 ppm). All concentrations quoted refer to the concentration of the solution before addition of the 50 μL of enzymes and reagents for analysis. Results indicate that the glucose calibration curves were relatively reproducible (FIG. 11). The limit of detection was ca. 0.3 ppm.

### EXAMPLE 9

### Tolerance of Glucose Assay to Synthetic Formation Water

To determine whether the glucose Amplex Red assay could tolerate synthetic formation water, two glucose solutions were prepared by diluting the stock solution (400 mM) to 18 ppm and 3.6 ppm with formation water. Results indicate that the assay tolerates the presence of formation water (FIG. 12)

### EXAMPLE 10

### Tolerance of Glucose Assay to Presence of Treatment Substances

To determine whether the glucose assay could tolerate the presence of treatment chemicals, the effects of scale inhibitor, corrosion inhibitor, isopropanol (IPA), methanol and monoethylene glycol were determined A 10% scale inhibitor solution was prepared by adding 100 μL of scale inhibitor 8017C to 100 μL of glucose (50 mM) and 800 μL of formation water. A 1% solution was prepared by adding 10 μL of scale inhibitor 8017C to 100 μL of glucose (50 mM) and 890 μL of formation water. Controls were prepared by the same method, substituting deionised water for the scale inhibitor. These samples were left at room temperature for 4 h and then serial diluted 1:10 twice, to give a final concentration of 50 μM glucose. 10% and 1% corrosion inhibitor EC1440A solutions were prepared in the same way.

Aqueous solutions of methanol, IPA and MEG (20%) were serial diluted 1:10 with water to give 2% and 0.2% solutions. A stock solution of 100 μM glucose was used. 1 mL glucose solution was added to 1 mL of each concentration of each solvent to give 12 samples with 10%, 1% and 0.1% final solvent concentration and 50 μM final glucose concentration. A control containing 1 mL water added to 1 mL glucose solution was also prepared.

The results can be seen in FIGS. 13 and 14. Scale inhibitor 8017C did not have any effects on the glucose assay. The presence of both 10% and 1% corrosion inhibitor EC1440A decreases the amount of glucose detected although the glucose concentration is well above that expected to be encountered in produced fluids (0.1% is considered a maximum amount expected). The assay is therefore effective in the presence of corrosion inhibitor and scale inhibitor.

### EXAMPLE 11

### Tolerance of Glucose Assay to the Presence of Additional Tracers

To determine if the glucose assay could function even in the presence of other tracers, so enabling multiple tracers to be used at once the effects of inclusion of biotin in the solution was determined The following four samples were prepared and analysed: 1) Water, 2) Biotin (0.5 μM), 3) Glucose (50 μM), Biotin (0.5 μM) and Glucose (50 μM). Results indicate that the assay tolerates the presence of biotin (FIG. 15).

### EXAMPLE 12

### Thermal and Acid Stability of Glucose

To determine the thermal and acid stability of glucose, 0.5 mM glucose solutions (10 mL) were prepared in both deionised water and formation water. These solutions were divided and the pH of one water sample and one formation water sample was adjusted to 2 with HCl. A 0.5 mL aliquot was removed from each sample before incubation to prepare control samples. The remaining 4.5 mL were placed in 4 duran bottles with Teflon tape wrapped around the threads to prevent evaporation. After heating at the required temperature (100, 120 or 150° C.) for 20 h, the bottles were cooled to room temperature and diluted 1:10 with deionised water.

Results are shown in FIG. 16. Samples heated to 100° C. showed no difference in detectability, although at 120° C. there was some evidence of degradation and at 150° C. samples showed a marked decrease in concentration compared to controls. These results indicate that glucose would be best applied to cooler systems, ideally those at or below 100° C. Incubation in solutions of pH 2 did not adversely impact glucose detection.

### EXAMPLE 13

### Tolerance of the Glucose Assay to Oil

To determine the effects of oil on the assay a 2% oil sample was prepared by adding 2% oil to 98% water by volume. The vial was shaken vigorously by hand and then serial diluted with water to ca. 0.2% and 0.02%. 50 μL of each oil concentration was added to 50 μL glucose solution (100 μM) to give final oil concentrations of 1%, 0.1% and 0.01%. The controls consisted of 50 μL of each oil concentration plus 50 μL water; as well as a 50 μL glucose solution (100 μM) plus 50 μL water sample.

Results (FIG. 17) indicated that as expected low levels of background fluorescence were observed for the oil plus water controls which increased with increasing concentration of oil. The assay, however, appeared unaffected indicating it could be used in oil-containing samples. Again, by first assessing background and then running the assay on the latently detectable glucose tracers, interfering background fluorescence can be removed.

Glucose is suitable for tracerling treatment substances, and for being detected within the context of an aqueous or organic solution. The limit of detection was ca. 0.3 ppm. The presence of oil, biotin, formation water, methanol, IPA, MEG and scale inhibitors did not have any significant effect on the levels of glucose detected by the assay. Glucose was found to be relatively stable at 100° C. however at 150° C. the concentrations detected were dramatically decreased. At 120° C. the pH 2 samples were stable while the glucose levels in the neutral samples dropped slightly. Corrosion inhibitors adversely affect the assay, even when present at very low concentrations.

### EXAMPLE 14

### Limits of Detection of Galactose

The general assay procedure for tests on galactose consisted of adding 50 μL of the solution to be analysed to 50 μL of working solution. 5 mL working solution was prepared from: 4.75 mL 1X reaction buffer, 100 μL galactose oxidase (100 U/mL), 100 μL horseradish peroxidise (10 U/mL), 50 μL amplex red or Amplex UltraRed (10 mM) (Invitrogen). Assays were carried out in 96-well plates and after addition of the working solution the plates were incubated at 37° C. for 30 min before analysis. The settings of the luminometer (Berthold Mithras) for analysis were as follows, lamp energy, 1000; λₑₓ 546 nm; λₑₘ 610 nm; counting time, 1 sec.

A calibration curve was generated by analysing galactose solutions prepared by serial dilution (50, 40, 30, 20, 10, 5, 2.5, 0.625, 0.3125, and 0 ppm). All concentrations quoted refer to the concentration of the solution before addition of the 50 μL of enzymes and reagents for analysis (FIG. 18A). To determine the reproducibility of the assay, these samples were rerun on three different days with freshly prepared working solution (FIG. 18B).

Results indicate that galactose can be detected within a concentration range of 0-30 ppm with a limit of detection of ca. 0.3 ppm. A linear response between 0 and 10 ppm is seen with R²=0.998. The assay was also shown to be reproducible; the graph displays the 95% confidence intervals. Further work suggested that Amplex Ultrared offered enhanced fluorescence and sensitivity and is recommended for use over Amplex Red reagent. Results indicate that galactose derivatives may be used to tag treatment chemicals, as they could also be detected with the assay (FIG. 19)

### EXAMPLE 15

### IMPACT OF INTERFERENCES ON GALACTOSE ASSAY

The effects of various potential interfering agents was investigated by preparing 2% aqueous solutions and then serial diluting to 0.2, 0.02, 0.002 and 0.0002%. Each of these solutions was added in a 50:50 ratio to 10 ppm galactose, therefore the final galactose concentration was 5 ppm. The interferences investigated using this method were scale inhibitors (2 types), a corrosion inhibitor, MEG, methanol and crude oil. Controls were prepared in which water was added in place of the galactose solution. Further controls for the scale and corrosion inhibitors and crude oil were run which did not contain any working solution (50 μL water was added instead), this was to determine the intrinsic fluorescence of these samples.

Results (FIG. 20) indicate that low concentrations of treatment chemicals (in concentrations expected in produced fluids e.g. <100 ppm scale inhibitor) do not adversely impact the assay.

### EXAMPLE 16

### Effect of Other Tracers on the Galactose Assay

To determine if the galactose assay could function even in the presence of other tracers, so enabling multiple tags to be used at once the effects of inclusion of fructose, mannose or glucose in the solution was determined Results indicate (FIG. 21) that fructose can be oxidized by galactose oxidase and would be unsuitable if used with galactose although mannose and glucose did not interfere with the assay and may be used as tracers in the same system.

### EXAMPLE 17

### Thermal Stability of Galactose and Derivatives

The thermal stability of both galactose and octy-galactose was investigated. Galactose and octyl-galacotse solutions (50 ppm, 30 mL) were prepared in both deionised water and formation water. These solutions were divided and the pH of one water sample and one formation water sample was adjusted to 2 with HCl. 4.5 mL of each solution was placed in a duran bottle with Teflon tape wrapped around the threads to prevent evaporation. The eight samples were heated at 100 or 120° C. for 20 h. The remaining solutions were kept at 4° C. inbetween experiments. Each of the samples was diluted 10-fold before analysis.

Results (FIG. 22) indicate that galactose and derivatives maybe sufficiently stable to 100° C. although a drop in concentration is observed above this temperature.

### EXAMPLE 18

### Limits of Detection for Xanthine and Hypoxanthine

An assay for determining the concentration of xanthine and hypoxanthine is commercially available. This assay uses xanthine oxidase to catalyze the oxidation of hypoxanthine or xanthine, to uric acid and superoxide. The superoxide spontaneously degrades to hydrogen peroxide (H₂O₂), which reacts stoichiometrically with Amplex® Red in the presence of horseradishperoxidase (HRP). A fluorescent product, resorufin, is generated which can be detected fluorometrically or spectrophotometrically. Results show that the limit of detection of xanthine is less than 0.16 ppm (FIG. 23) and the limit of detection of hypoxanthine is >0.02 ppm (FIG. 24).

### EXAMPLE 19

### Effect of Interferences on the Xanthine and Hypoxanthine Assay

The effects of various potential interfering agents was investigated by preparing 2% aqueous solutions and then serial diluting to 0.2, 0.02, 0.002 and 0.0002%. Each of these solutions was added in a 50:50 ratio to 12.5 ppm hypoxanthine, therefore the final hypoxanthine concentration was 6.25 ppm. The interferences investigated using this method were two scale inhibitors, a corrosion inhibitor, MEG, methanol and crude oil. Controls were prepared in which water was added in place of the hypoxanthine solution. Further controls for the scale and corrosion inhibitors and crude oil were run which did not contain any working solution (50 μL water was added instead), this was to determine the intrinsic fluorescence of these samples.

Corrosion inhibitor and methanol had an adverse affect on the assay at the highest concentrations investigated (10,000 ppm); however these levels are well above those expected in a real system (FIG. 25)

### EXAMPLE 20

### Thermostability of Xanthine and Hypoxanthine

The thermal stability of both xanthine and hypoxanthine was investigated; 75 ppm solutions were prepared in deionised water. These solutions were divided and the pH of one sample of each compound was adjusted to 2 with HCl. 4.5 mL of each solution was placed in a duran bottle with Teflon tape wrapped around the threads to prevent evaporation. The samples were heated at 120° C. for 20 h. The remaining solutions were kept at 25° C. Each of the samples was diluted 10-fold to 7.5 ppm before analysis. Results (FIG. 26) indicate that xanthine and hypoxanthine are stable at room temperature and 120° C. at both acidic and neutral pH.

1. A tracer for monitoring flow through a system for conduction and containment of fluid, wherein the interaction between the tracer and a biomacromolecule produces a detectable signal.

2. A tracer according to claim 1 wherein the biomacromolecule includes a site for specific interaction with the tracer.

3. A tracer according to claim 1 wherein the biomacromolecule and the tracer associate as part of molecular signalling complexes in nature.

4. A tracer according to claim 1, wherein the detectable signal is an optical signal.

5. (canceled)

6. A tracer according to claim 1, wherein the tracer is a small molecule that is known to interact with a specific biomacromolecule in nature.

7. A tracer according to claim 1, wherein the tracer comprises vitamins including biotin, selenobiotin or oxybiotin, thiamine, riboflavin, niacin (nicotinic acid), pathothenic acid, citrate, cobalamin, folic acid, ascorbic acid, retinol, vitamins C, D, E or K; luciferin; coelenterazine; chitin; amino acids such as histidine; or monosaccharides, polysaccharides and carbohydrates including arabinose, deoxyribose, lyxose, ribulose, xylose, xylulose, maltose, glucose, fructose, ribose, or trehalose, caffeine, imidazoline, steroid hormones, chlorpromazine and cAMP, cortisol, 6-ketoprostaglandins, hyroxine, triiodothyronine, anthocyanins, cholesterol, L-gulono-1,4-lactone, bile salts including cholic acid, chenodeoxycholic acid, deoxycholic and glycocholate eicosanoids (prostaglandins, prostacyclins, the thromboxanes and the leukotrienes), galactose and derivatives including 2-N-acetyle galactose, 1-methyl-beta-D-galactose, 1-octyl-beta-D-galactose, xanthine and hypoxanthine, catchetolamines such as epinephrine and norepinephrine, nucleotides such as adenine, cytosine, guanine, tyrosine, uracil, monophosphate, in diphosphate and triphosphate forms and the associated biomacromolecule is selected accordingly to the tracer used from; avidin and its functional analogues e.g. streptavidin, neutravidin and nitroavidin; thiamine binding-protein; riboflavin binding protein (flavoprotein); nicotinic acid binding protein; pantothenic acid binding protein; citrate binding protein, cobalamin binding protein; folic acid binding protein; ascorbic acid binding protein; retinol binding protein; vitamin D binding protein e.g. group specific protein (Gc); Vitamin E binding protein; Vitamin K binding protein; luciferase; coelenterate luciferase; chitin binding protein; histidine transporter protein; arabinose binding protein; deoxyribose binding protein; lyxose binding protein; ribulose binding protein; xylose binding protein; xylulose binding protein; maltose binding protein; glucose binding protein; fructose binding protein; ribose binding protein; trehalose binding protein or lectin; caffeine binding protein; imidazoline binding protein; steroid hormone receptors; chlorpromazine binding protein; cAMP binding protein; cortisol binding protein; 6-keto-prostaglandin antibody including labelled antibodies such as aqueorin or GFP labelled antibodies; thyroxine binding proteins including thyroxine-binding globulin, transthyretin and albumin; triiodothronine binding protein; glutathione-S-transferases; cholesterol binding proteins such as VIP21/caveolin and cholesterol oxidase; L-gulono-1,4-lactone binding proteins including Rv1771, L-gulono-1,4-lactone dehydrogenase and L-gulono-1,4-lactone oxidase; glutathione S-transferases and bile binding proteins including ileal bile acid binding proteins and liver fatty acid-binding proteins, prostaglandin receptors including PPARg, prostacyclin receptors including PTGIR and thromboxane receptors such as TXA2; L-ascorbate binding protein including L-ascorbate oxidase; galactose binding protein including galactose oxidase, xanthine oxidase, xanthine dehydrogenase, phosphoribosyltransferase, xanthine binding RNAs, catecholamine regulated protein (CRP40), catecholamine binding proteins, adrenergic receptors (alpha and beta), epinephrine receptor, norepinephrine receptor; nucleotide binding proteins such as G proteins and ATP binding proteins respectively.

8. A tracer according to claim 1, wherein the tracer is associated with at least one treatment substance, the treatment substance being used to maintain efficient flow within a fluid system.

9-12. (canceled)

13. A method of monitoring the flow of fluid through a system for the conduction and containment of fluid comprising:
a) adding a predetermined amount of at least one tracer for monitoring flow through a system for conduction and containment of fluid, wherein the interaction between the tracer and a biomacromolecule produces a detectable signal, at a first location in the system;
b) adding a biomacromolecule according to claim 7 to the fluid in at least second location in said system, said second location being downstream of said first location, wherein the predetermined amount of the detectable tracer at the first location is sufficient for the concentration of the detectable tracer at the second location to be above its detection limit of 1 ppb, the concentration of the associated biomacromolecule being sufficient to produce a detectable change in the fluid due to a specific interaction of the detectable tracer with the biomacromolecule;
c) detecting the change in the fluid;
d) analysing the measured detectable change to determine the concentration of the tracer at the second location;
e) using the data obtained in step (d) to assess flow characteristics of the fluid in the fluid conducting and containment system

14. A method according to claim 13, further comprising the step of taking a sample of fluid from the second location.

15. A method according to claim 14, further comprising the step of treating the sample to improve detectability of the signal, such that the sample is concentrated, bleached, filtered or immobilised to improve detectability of the signal.

16. (canceled)

17. (canceled)

18. A method according to claim 13, further comprising the step of adding a second molecule before detecting the change in the fluid.

19. A method according to claim 18, wherein the second detection molecule reacts with a chemical product of the interaction between the tracer and the biomacromolecule, and wherein the chemical product is hydrogen peroxide.

20. (canceled)

21. A method according to claim 18, wherein the second detection molecule is Amplex Red in the presence of peroxidase; Phenol Red in the presence of peroxidase; ferrous ions in the presence of xylenol or orange; or a cyclic diacy hydrazide in the presence of peroxidase.

22. A method according to claim 13, wherein multiple tracers are monitored.

23. A method according to claim 13 wherein the tracer is detectable, in the presence of said biomacromolecule, by a fluorescence detector, luminescence detector, Raman detector, optical microscope, CCD camera, photographic film, fibre-optic device, photometric detector, MEMS device, single photon detector, spectrophotometer, chromatography system or by eye.

24. A method according to claim 13, wherein the detection of tracers is carried out offline, inline, atline or online.

25-27. (canceled)

28. A method according to claim 13, wherein the tracer is associated with a treatment substance.

29-31. (canceled)

32. A kit for use in monitoring the flow of fluid through a system for conduction and containment of fluid, comprising;
a) a tracer for monitoring flow through a system for conduction and containment of fluid, wherein the interaction between the tracer and a biomacromolecule produces a detectable signal and
b) a biomacromolecule selected accordingly to the tracer included in the kit.

33. A kit according to claim 32, further including means for taking a sample from said system.

34. A kit according to claim 32, further including a second detection molecule.

35. (canceled)
